(19) United States
(12) Patent Application Publication
(10) Pub. No.: US 2011/0008814 A1
(43) Pub. Date: Jan. 13, 2011

(54) Enzyme Immunoassay Using Enzyme-Labeled Antibody

(75) Inventor: Shigeru KOGUCHI, Noda-shi (JP)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: KIKKOMAN CORPORATION, Chiba (JP)

(21) Appl. No.: 12/834,107

(22) Filed: Jul. 12, 2010

(30) Foreign Application Priority Data
Jul. 13, 2009 (JP) 2009-164343

(51) Int. Cl. C12Q 1/28; C12N 9/04

(52) U.S. Cl. 435/28; 435/190

(57) ABSTRACT

It was able to provide a method for carrying out an enzyme immunoassay such as ELISA or sandwich ELISA, which hardly inhibited by antiseptics and has high reactivity and sensitivity similar or superior to the performance in the case of the use of an antibody labeled using POD derived from horseradish, by carrying out the enzyme immunoassay by labeling a POD derived from Basidiomycetes and using the labeled antibody under an immobilized state. Additionally, even when antiseptics including sodium azide are added, the POD activity is hardly inhibited so that it was able to carry out the enzyme immunoassay with good reactivity and sensitivity.

### TECHNICAL FIELD

This invention relates to an enzyme immunoassay which uses an enzyme-labeled antibody. Specifically, it relates to an enzyme immunoassay which uses POD-labeled antibody obtained by labeling a peroxidase (POD) derived from Basidiomycetes with an antibody or a fragmentation antibody such as IgG and/or Fab′.

### BACKGROUND OF THE INVENTION

Conventionally, enzyme immunoassay is known as a technique for measuring trace amount of an antigen, antibody or the like. As the technique of enzyme immunoassay, for example, enzyme-linked immunosorbent assay (ELISA), western blotting and the like are broadly used (e.g., see Patent Reference 1).

A POD derived from a horseradish is broadly used as one of the enzymes used in the above-mentioned various types of enzyme immunoassay. However, since the POD derived from horseradish is derived from a plant, it is difficult to carry out its mass production by a microorganism using recombinant DNA techniques. Accordingly, under such a condition that amount of POD contained in horseradish cannot be said sufficient, POD is produced by disrupting the plant body and purifying it from a great variety of impurities, so that it cannot be said that the production efficiency is high. There is also a problem that a prolonged period of time is required for cultivating horseradish as the material. Furthermore, because of the poor cultivation efficiency, change of cultivation to large demand crops for bio-ethanol, and the like in recent years, there is a possible situation of generating uncertainty for the supply of horseradish as the material of POD. Therefore, potential needs for an enzyme substitutable for it are great.

Additionally, there also is a problem that many isozymes are present in the POD derived from horseradish. Because of the limitation that it must be circulated at a certain cost regardless of the low production efficiency as described in the above, it is the most case that many of the POD articles derived from horseradish currently appearing on the market are mixtures of many isozymes. However, when various types of measurement, such as an enzyme immunoassay, is carried out using such a POD, containing amounts of various isozymes having different reaction characteristics vary in each production lot of POD, and caused by this, which causes a serious problem in that it becomes difficult to obtain a stable measuring result.

There is a microbial POD as a POD which could solve the aforementioned problems possessed by the POD derived from horseradish. Since microorganisms can be cultured in a large amount within a short period of time, a microbial POD can be purified with markedly smaller troubles than the case of purifying from a plant body. Additionally, it also is easy to artificially increase expression level in a microbial host by the use of recombinant DNA techniques. Since the POD of interest alone can be expressed in a large amount when recombinant DNA techniques are used, the problem of contamination of isozymes can be easily avoided. At the same time, it is relatively easy to carry out alteration and modification of the POD. Because of these, a microbial POD is a promising enzyme which can be replaced with the POD derived from horseradish.

However, there is little information in which the performance of enzyme immunoassay was examined using a microbial POD. As a conventionally known microbial POD, there is a known POD derived from the genus Arthromyces, and it has been reported that its reactivity is superior to that of the POD derived from horseradish, in a free state or in a measuring system in which a labeled antibody is prepared by binding to an antibody and the labeled antibody is used in a state of being fixed to a solid layer via a substance to be measured (e.g., see Patent Reference 2 and Non-patent Reference 1). Also, it has been reported that the reactivity is higher than the case of the use of a horseradish-derived POD-labeled antibody, in an ELISA system which uses an antibody labeled with POD derived from Arthromyces ramosus using the periodic acid method (e.g., see Non-patent Reference 1). However, the highness of the reactivity of POD is limited to about 4 times of the case of using the horseradish-derived POD-labeled antibody, and it has been reported that there is a problem on the heat resistance of the POD.

Additionally, the inventors of the present invention have prepared a POD derived from genus Coprinus (e.g., see Patent Reference 3 and Non-patent Reference 2), and by using it under a free form, have made an attempt to compare its performance with the reactivity of the POD derived from horseradish in a coloring reaction system or luminous reaction system. As a result, when it was used under a free state, the enzyme showed approximately from 9 to 10 times higher activity than that of POD derived from horseradish by a coloring reaction using 2,2′-azinobis(3-ethylbenzothiazoline-6-sulfonic acid) (ABTS) as the substrate. Additionally, in a luminous reaction system which uses luminol as the substrate, a luminescence equivalent to the POD derived from horseradish to which an activator, p-iodophenol, was added was observed. However, when an antibody was labeled with the genus POD derived from Coprinus and the POD-labeled antibody was used under a free state in the luminous reaction using luminol as the substrate, contrary to the above-mentioned tendency, it was confirmed that the reactivity was inferior to that of POD derived from horseradish.

Namely, concern has been directed toward the provision of an enzyme immunoassay which has equivalent or higher reactivity and sensitivity than the case of the use of POD derived from horseradish, without using the POD derived from horseradish in various types of enzyme immunoassay in which a labeled antibody is used under an immobilized state, such as the case of ELISA, sandwich ELISA, western blotting and the like.

Additionally, since the enzyme-labeled antibody solution to be used in the above-mentioned enzyme immunoassay is generally apt to rot, it is necessary to add an antiseptic agent for preservation. Various types of such an antiseptic agent are known, and particularly, sodium azide and the like are broadly used because of their excellent antiseptic effect at a neutral pH range. However, it is known that sodium azide strongly inhibits the activity of POD derived from horseradish even when added in a trace amount (e.g., see Non-patent Reference 3). In order to solve the problem, attempts have been made to reduce inhibition of the peroxidase activity by sodium azide, for example, by adding a polyalkylene glycol (e.g., see Patent Reference 4) or phenol compounds (e.g., see Patent Reference 5) under coexistence of a peroxidase labeling substance and sodium azide. Also, a method for stabilizing the POD activity by adding horseradish POD to solution containing a POD labeling substance has been reported (e.g., see Patent Reference 6). However, when an additive unrelated to the original measuring reaction is added intentionally to the measuring system of enzyme immunoassay, it has a possibility of exerting useless influences upon the antigen-antibody reaction and various substances to be measured. Additionally, the method for adding POD derived from horseradish is limited to the method in which B/F separation (an operation to separate an antigen binding to an antibody from the antigen which does not bind to the antibody) is carried out, so that it has a problem in that the application range is limited.

Because of the above-mentioned situation, concern has been directed toward an enzyme immunoassay in which the POD activity is hardly inhibited even when antiseptics such as sodium azide and the like are used, without adding an additive substance unrelated to the original measuring reaction, namely which is excellent in antiseptics resistance.

### Related Art References

[Patent References]

[Patent Reference 1] JP-A-2000-88850

[Patent Reference 2] Japanese Patent No. 2528457

[Patent Reference 3] JP-A-3-1949

[Patent Reference 4] JP-A-2-138999

[Patent Reference 5] JP-A-7-135975

[Patent Reference 6] JP-A-7-222600

[Non-Patent References]

[Non-patent Reference 1] Kim et al., Analytical Biochemistry, 199, 1-6 (1991)

[Non-patent Reference 2] Kjalke et al., Biochim. Biophys. Acta. 1992 Apr. 17; 1120 (3): 248-56

[Non-patent Reference 3] Maehly et al., Methods in Enzymology, vol. II, pp. 801-813 (1969)

### SUMMARY OF THE INVENTION

A problem of the present invention is to provide a method which can carry out an enzyme immunoassay such as ELISA and the like which has equivalent or superior reactivity and sensitivity in comparison with the performance in the case of the use of an antibody labeled with POD derived from horseradish. Furthermore, it is to provide a method which can carry out an enzyme immunoassay such as ELISA and the like with good reactivity and sensitivity, in which the POD activity can hardly be inhibited even when antiseptics such as sodium azide and the like are added. The problem of the present invention is solved by the following.

(1) An enzyme immunoassay, which comprises measuring a coloring level or emission level derived from a peroxidase reaction by an antibody or fragmentation antibody which is labeled with a peroxidase derived from Basidiomycetes, wherein said coloring level or emission level is increased in comparison with the case of using an antibody or fragmentation antibody which is labeled with a peroxidase derived from horseradish.

(2) The enzyme immunoassay according to (1), wherein the antibody or fragmentation antibody is immobilized.

(3) The enzyme immunoassay according to (1), wherein the Basidiomycetes is a microorganism belonging to the genus Coprinus.

(4) The enzyme immunoassay according to (1), wherein the peroxidase derived from Basidiomycetes is a polypeptide having an amino acid sequence wherein at least one of deletion, addition, insertion and substitution is made on one or two or more amino acid residues of the amino acid sequence described in SEQ ID NO:2.

(5) The enzyme immunoassay according to (1), wherein the peroxidase derived from Basidiomycetes is a polypeptide having an amino acid sequence described in SEQ ID NO:2.

(6) The enzyme immunoassay according to (1), wherein the peroxidase derived from Basidiomycetes or an antibody or fragmentation antibody labeled with the peroxidase derived from Basidiomycetes has an antiseptic resistance.

(7) The enzyme immunoassay according to (6), wherein the antiseptic is proclin, thimerosal, sodium azide, gentamicin or a derivative thereof.

(8) A composition for enzyme immunoassay, which comprises an antibody labeled with a peroxidase derived from genus Coprinus, wherein the aforementioned labeled antibody carries out a coloring reaction or emission reaction under an immobilized state.

(9) The composition for enzyme immunoassay according to (8), wherein the peroxidase derived from genus Coprinus or an antibody or fragmentation antibody labeled with the peroxidase derived from genus Coprinus has an antiseptic resistance.

(10) The composition for enzyme immunoassay according to (9), wherein the antiseptic is proclin, thimerosal, sodium azide, gentamicin or a derivative thereof.

(11) A kit for enzyme immunoassay, which comprises the composition for enzyme immunoassay described in (8).

(12) An antibody or fragmentation antibody labeled with a peroxidase derived from a Basidiomycete belonging to the genus Coprinus.

(13) The antibody or fragmentation antibody according to (12), which is a polypeptide having the amino acid sequence described in SEQ ID NO:2.

### BRIEF DESCRIPTION OF THE DRAWINGS

FIG. 1: A figure showing reactivity (secondary antibody concentration-coloring level) in the detection of mouse IgG (primary antibody) by ELISA (coloring system). (A) shows a test result of Comparative Example 1, and (B) shows that of Comparative Example 2 and (C) shows that of Example 1, respectively, showing measured results when concentration of mouse IgG is 100 ng/ml, 10 ng/ml or 1 ng/ml. Longitudinal axes indicate absorbance at 405 nm.

FIG. 2: A figure showing reactivity (human transferrin concentration-coloring level) in the measurement of human transferrin by sandwich ELISA (coloring system). (A) shows a test result of Example 3, and (B) shows that of Comparative Example 3, (C) shows that of Comparative Example 4 and (D) shows that of Comparative Example 5, respectively.

FIG. 3: A figure showing reaction time and coloring level in the measurement of human transferrin (1 ng/ml) by sandwich ELISA (coloring system).

FIG. 4: A figure showing reaction time and coloring level in the measurement of human transferrin (100 ng/ml) by sandwich ELISA (coloring system).

FIG. 5: A figure showing reactivity (human transferrin concentration-coloring level) in the measurement of human transferrin by sandwich ELISA (coloring system). (A) shows a test result of Comparative Example 6, and (B) shows that of Comparative Example 7, (C) shows that of Comparative Example 8, (D) shows that of Example 5 and (E) shows that of Example 6, respectively.

FIG. 6: A figure showing reactivity (α2-macroglobulin concentration-coloring level) in the measurement of α2-macroglobulin by sandwich ELISA (coloring system).

FIG. 7: A figure showing reactivity (POD-labeled antibody concentration-coloring level) in the measurement of α2-macroglobulin by sandwich ELISA (coloring system).

FIG. 8: A figure showing detection of human-derived plasma and human transferrin using western blotting (P: human-derived plasma, T: human transferrin, M: molecular weight marker). (A) shows a test result when POD-IgG was used, and (B) shows that when the POD-Fab′ of the present invention was used, respectively.

FIG. 9: A figure showing immunostaining with an anti-actin antibody of HeLa cell in the case of using the POD-Fab′ of the invention. (A) shows case of 1/2000 dilution and (B) shows 1/10000 dilution respectively.

FIG. 10: A figure showing immunostaining with an anti-insulin antibody of rat pancreas. (A) shows a case of 1/1000 dilution using POD-IgG (33 μg/ml), and (B) a case of 1/1000 dilution using POD-Fab′ (12 μg/ml).

FIG. 11: A figure showing reactivity (sodium azide concentration-residual activity) in the detection of mouse IgG (primary antibody) by ELISA (coloring system) using a POD-labeled antibody which was allowed to coexist with sodium azide for a certain period of time.

FIG. 12: A figure showing reactivity (concentration of each antiseptic-residual activity) in the detection of mouse IgG (primary antibody) by ELISA (coloring system) using a POD-labeled antibody which was allowed to coexist with each antiseptic for a certain period of time.

### DETAILED DESCRIPTION OF THE INVENTION

With the aim of solving the above-mentioned problems, the inventors of the present invention have conducted intensive studies and found as a result that when an antibody is labeled with a POD derived from Basidiomycetes and an enzyme immunoassay such as ELISA and the like is carried out using this labeled antibody, it is able to obtain reactivity and sensitivity higher than the reactivity and sensitivity in the case of using a POD derived from horseradish. Additionally, it was found that when an enzyme immunoassay such as ELISA and the like is carried out using the labeled antibody, the POD activity is hardly inhibited and an enzyme immunoassay such as ELISA and the like can be carried out with good reactivity and sensitivity even when various antiseptics including sodium azide are added to accomplish the invention.

The following describes modes for carrying out the invention.

### Microbial POD to be Used in the Invention

As the POD to be used in the enzyme immunoassay of the invention, a POD derived from Basidiomycetes can be mentioned. Examples of the microorganisms belonging to Basidiomycetes include the genus Coprinus, the genus Uredinales, the genus Auriculariales, the genus Agaricales and the like. Of these, although examples of the microorganisms belonging to the genus Coprinus include Coprinus cinereus (NBRC 30114), Coprinus macrorhizus (ATCC 20120), Coprinellus disseminatus, Coprinus comatus (ATCC 12640), Coprinus clastophyllus, Coprinus alkalinus, Coprinus amphibius, Coprinus micaceus, Coprinus atramentarius, Coprinus luteocephalus, Coprinus trisporus, Coprinus sclerotiger, Coprinus domesticus, Coprinus stercorarius, Coprinus radiatus and the like, the microorganisms are not limited thereto. In this connection, NBRC represents Biological Resource Center, National Institute of Technology and Evaluation, and ATCC represents American Type Culture Collection.

Additionally, even in the case of microorganisms which do not belong to the genus Coprinus, a POD derived from a microorganism relative to the genus Coprinus, a POD which has an amino acid sequence close to that of the POD derived from the genus Coprinus and shows similar reactivity in the enzyme immunoassay of the present invention, and the like are also included in the POD usable in the enzyme immunoassay of the invention. An example of gene sequence of a POD derived from the genus Coprinus (Coprinus cinereus) is shown in SEQ ID NO:1, and an example of amino acid sequence of the same in SEQ ID NO:2.

As examples of the gene encoding the POD of the present invention include a gene obtained by cloning said gene from a microorganism capable of producing said enzyme and a gene having a homology with said gene. Regarding the homology, a gene having a homology of at least 60% or more can be used and a gene having a homology of 90% or more is preferable. A gene having a homology of 95% or more is more preferable and a gene having a homology of 98% or more is most preferable.

Regarding the amino acid sequence of POD, a polypeptide having an amino acid sequence in which at least one of deletion, addition, insertion and substitution is made on one or two or more amino acid residues is preferable. Furthermore, a polypeptide having the amino acid sequence described in SEQ ID NO:2 of the SEQUENCE LISTING is more preferable.

The POD may be natural products, those into which one or more of certain mutations, such as improvement of heat resistance, improvement of substrate specificity and the like, are introduced, or chimeric proteins and the like. Additionally, POD may be used by purchasing a commercially available POD. An enzyme purified into uniformity can also be used, or POD having a different origin, structure or function and two or more POD including isozymes may be used together within such a range that there is no difficulty in carrying out measurement.

### Antibody

The antibody to be used in the enzyme immunoassay of the present invention may be any immunoglobulin class and subclass and further may be an antibody fragment. The antibody fragment means a part of the aforementioned antibody, and illustratively, F(ab′)₂, Fab′, Fab, Fv, Disulphide-linked Fv, single chain Fv (scFv) and polymers thereof, and the like corresponds to it. Additionally, it may be a polyclonal antibody or monoclonal antibody.

### Enzyme Immunoassay

The enzyme immunoassay of the present invention may be a competitive assay which uses an antibody or a noncompetitive assay. Also, it may be a homogenous assay (measurement by a homogenous system) or a heterogeneous assay (measurement by a heterogeneous system). For example, the enzyme immunoassay of the present invention can be carried out in accordance with a conventional method such as an enzyme immunoassay (EIA), an enzyme-linked immunosorbent assay (ELISA), an ELISPOT method, an immunoblotting, a western blotting, an immunostaining and the like.

As a suitable specific method of assay which uses the antibody of the present invention, ELISA can for example be mentioned. These methods are methods which use an antibody or antigen labeled with an enzyme and amount of the antibody or antigen is determined by the activity of the enzyme (in general, this is converted into coloring level, emission level and the like in most cases). Specific procedures of respective methods are well known to those skilled in the art and, for example, the methods described in the examples which are described later in the present specification can be used.

### Immobilization of Labeled Antibody

In the enzyme immunoassay of the present invention, immobilized antibody and antigen are used in order to separate a labeled antigen-antibody combination and free type labeled antigen or antibody. As the solid phase, agarose, inner surface of microtiter plate, latex particles, beads of various materials, and the like can be used. Illustrative procedures of respective methods are well known to those skilled in the art. For example, the methods described in the examples which are described later in the present specification can be used.

The term “immobilized state” as used in the enzyme immunoassay of the present invention does not always means that the POD-labeled antibody itself is directly immobilized on a microtiter plate or bead. For example, it may be bound with an immobilized antigen via an antibody like the case of ELISA, or an antigen may be bound to an immobilized antibody and then immobilized under a state of recognizing the antibody like the case of sandwich ELISA. Additionally, POD may be bounded with avidin or streptoavidin and then immobilized via biotin. Namely, regarding a state under which an antibody labeled with POD is indirectly immobilized on a solid phase via a certain intervening molecule, it is also included in the “immobilized state” of the enzyme immunoassay of the present invention.

### Labeling of Antibody Using POD

As the labeling (crosslinking) method of an antibody using POD to be used in the present invention, conventionally known various labeling methods can be used. Examples of generally known methods include glutaraldehyde method, periodate method, maleimide method, pyridyl disulfide method, isocyanate crosslinking method, benzoquinone crosslinking method and the like. The maleimide method is particularly desirable from the viewpoint of the presence or absence of polymer formation, activity keeping of the antigen, antibody and enzyme and, further from the viewpoint of the labeling efficiency.

In the maleimide method, maleimide group is introduced into POD using a compound having a maleimido group and a succinimide ester group in one molecule. Examples of the reagent for this include a divalent crosslinking agent having maleimido group in one end and N-hydroxysuccinimido group on the other end. Specifically, although the Examples include N-(6-maleimidocaproyloxyl)succinimide (EMCS), N-(4-maleimidobutyloxy)succinimide (GMBS), N-succinimidyl-N-maleimide acetate, N-succinimidyl-4-(N-maleimide) butyrate, (N-succinimidyl-4-(N-maleimido)butyrate), N-succinimidyl-6-(maleimido)hexanoate, (N-succinimidyl-6-(N-maleimido)hexanoate, N-succinimidyl-4-(N-maleimidomethyl)cyclohexane-1-carboxylate, N-sulfosuccinimidyl-p-(N-maleimidophenyl)-4-butyrate and the like, the reagent is not limited thereto. These substances form acid amide bond (—NH—CO—) by bonding with amino group of the enzyme and simultaneously introduce maleimido group. As is described later, the introduced maleimido group binds to a carrier by reacting with thiol group in the antibody and thereby forming thioether bond (—S—).

### Coloring Reaction and Emission Reaction

According to the enzyme immunoassay of the present invention, an emission system and/or coloring system can be constructed together with a substance which becomes the reaction substrate of the POD which labeled the antibody. Examples of the emission system reagent include luminol, NovaLume, L-012 (Wako Pure Chemical Industries) and the like. Although examples of the coloring system reagent include 2,2′-azinobis(3-ethylbenzothiazoline-6-sulfonic acid) (ABTS, mfd. by Roche), 3,3′,5,5′-tetramethylbenzidine (TMBZ), diaminobenzidine (DAB), HistoMark BLACK (mfd, by Funakoshi), HistoMark Orange (mfd, by Funakoshi), HistoMark TrueBlue (mfd, by Funakoshi) and the like, the reagent is not limited thereto.

Although a good measuring result can be obtained by the use of each of the emission system and/or coloring system, in recent years of demanding for further high sensitivity, there is a tendency in that an assay system making use of a principle of the emission system is used more preferably.

In this connection, in the enzyme immunoassay which uses a POD derived from horseradish, addition of an activator which sharply improves the reactivity of POD (e.g., see p-iodophenol: Coyle et al., Ann. Clin. Biochem., January 1986, 23 (Pt. 1): 42-6) to the reaction system is generally carried out as a technique for improving measuring sensitivity. The reactivity reinforcing activity of the activator is specific for the POD derived from horseradish, and the reinforcing effect does not occur in a microbial POD. Accordingly, in carrying out screening of a microbial POD capable of substituting POD derived from horseradish, it is more desirable that the POD derived from horseradish in the state of being mixed with the activator is used as the standard and an enzyme equivalent or superior to that is screened. Alternatively, a new activator capable of improving the reactivity of microbial POD may be screened and used in combination.

Evaluation of the emission level and/or coloring level can be carried out based on the emission level just after commencement of the reaction, and/or based on the integrated value of the emission level or coloring level during the measuring period of time, and the like. According to the enzyme immunoassay of the present invention, an emission level (reactivity) markedly exceeding the POD derived from horseradish in the state of being mixed with the activator can be obtained when an antibody labeled with the genus Coprinus-derived POD is used and used in the enzyme immunoassay under such a sate that the labeled antibody is immobilized. Although the tendency is the same also in the case of using the coloring method, its superiority is significant particularly in the emission system.

### Composition for Enzyme Immunoassay

The POD, and/or the antibody labeled with the aforementioned POD, to be used in the enzyme immunoassay of the present invention can be provided as a general purpose reagent for studies, but can also be provided as a composition for enzyme immunoassay, which contains a coloring system or emission system reagent and, as occasion demands, other components for preparing a composition optimized for improving sensitivity of the assay system by reinforcing the coloring/emission strength, such as the components which contribute to the preservation stability of the enzyme, including a buffer, a metal and/or an antiseptic, and the like.

### Antiseptic Resistance

Feature of the POD, and/or the antibody labeled with the aforementioned POD, to be used in the enzyme immunoassay of the present invention and contained in the composition for the enzyme immunoassay of the present invention is that they show a certain level or more of antiseptic resistance when an antiseptic is coexist in the composition for enzyme immunoassay. Although kinds of the antiseptic are not particularly limited, examples of the antiseptic include proclin, thimerosal, sodium azide, gentamicin and derivatives thereof. The term “shows antiseptic resistance” means that even when one or more antiseptics having a suitable concentration is coexist in the enzyme immunoassay, it shows a degree of POD activity equal to the case without the aforementioned antiseptics. Alternatively, although it means that the POD activity is lowered in comparison with the case without the aforementioned antiseptics, the lowering degree of POD activity is markedly small in comparison with various kinds of conventionally known PODs and/or antibodies labeled with various kinds of conventionally known PODs.

As a suitable example of the composition for enzyme immunoassay of the present invention, even when 1% sodium azide is coexisted, the POD activity is not lowered in comparison with the case without the aforementioned antiseptics, which shows possession of excellent antiseptic resistance. Additionally, as a suitable example of the composition for enzyme immunoassay of the present invention, even when an antiseptic is coexisted, it shows a POD activity of 50% or more in case without the antiseptic. It is preferable that the activity is 60% or more; it is more preferable that the activity is 70% or more; it is further preferable that the activity is 80% or more; and it is most preferable that that the activity is 90% or more, in case without the antiseptic.

### Kit for Enzyme Immunoassay

Although The POD, and/or the antibody labeled with the aforementioned POD, to be used in the enzyme immunoassay of the present invention can be provided as a general purpose reagent for studies, it can also be provided as a kit for enzyme immunoassay, which comprises two or more compositions for enzyme immunoassay containing a coloring system or emission system reagent and, as occasion demands, other components for preparing a composition optimized for improving sensitivity of the assay system by reinforcing the coloring/emission strength, such as a buffer, a metal and/or a components which contribute to the preservation stability of the POD, and the like; and/or solution for dilution of samples; and/or a micro-well plate for assay; tools for operation; a manual and the like in combination as a set.

The following describes the present invention further specifically based on examples. However, the technical scope of the present invention is not limited by the Examples.

### Example 1

### POD Labeling of Anti-Mouse IgG Antibody

### 1) Preparation of F(ab′)₂

For a goat anti-mouse IgG (H+L) antibody (mfd. by Bethyl, A90-116A) (3 to 5 mg/ml), solution substitution was carried out to 0.1 M sodium acetate buffer (pH 4.0). A 1/50 volume (w/w) of a pig stomach-derived pepsin (mfd. by Sigma, P7012) was added thereto and incubated at 37° C. for 20 hours. Thereafter, the reaction was stopped by adding 1/10 volume (v/v) of 3.0 M Tris-HCl (pH 8.8). It was applied to a cellulose resin DE52 (4057-200 mfd. by Whatman, equilibration: 10 mM Tris-HCl (pH 8.0), eluent: 10 mM Tris-HCl (pH 8.0) containing 50 mM NaCl, column size: 10 ml, elution by gravity), which had been equilibrated with 50 mM NaCl and 20 mM Tris-HCl (pH 8.5), and the passed through fractions were recovered. Concentration of the F(ab′)₂ antibody recovered in the passed through fractions was calculated from the absorbance measured value at 280 nm using ε280 nm=1.481/(g·cm).

### 2) Preparation of Fab′₂(IgG)-SH, IgG-SH

For a goat anti-mouse antibody (F(ab′)₂, or IgG), solution substitution was carried out to 0.1 M sodium phosphate (pH 6.4), 0.15 M NaCl and 10 mM EDTA and adjusted to a concentration of from 1 to 10 mg/ml. By preparing 500 mM cysteamine (mfd. by Sigma, M9748-25G (6 mg/0.1 ml of 0.1 M sodium phosphate (pH 6.4), 0.15 M NaCl, 10 mM EDTA), 1/10 volume (v/v) was added to the aforementioned antibody solution and incubated at 37° C. for 1.5 hours. Excess cysteamine was removed by dialysis or a gel filtration column Sephadex G-25 (mfd. by GE, 17-0033-01, equilibration liquid: 0.1M sodium phosphate (pH 6.8), 0.15 M NaCl, 10 mM EDTA, eluent: 0.1 M sodium phosphate (pH 6.8), 0.15 M NaCl, 10 mM EDTA, column size: 10 ml, elution by gravity).

### 3) Preparation of Maleimidation POD

A commercially available POD (mfd. by Roche) was suspended in 0.1 M sodium phosphate (pH 7.4), 0.15 M NaCl and 10 mM EDTA to a concentration of from 10 to 30 mg/ml. A 100 mM SMCC (mfd. by Pierce, 22360) (dissolved in 3.34 mg/0.1 ml DMSO) was prepared and immediately 5 times amount of mol concentration of POD was added. It was incubated at room temperature for 30 minutes and subjected to a gel filtration column Sephadex G-25 in accordance with the aforementioned conditions, and excess SMCC was removed. Oxygen concentration was calculated from the absorbance measured value at 403 nm using ε403 nm=8.33×10⁴ l/(mol·cm).

In this connection, enzymological properties of the above-mentioned commercially available POD when ABTS (2,2′-azinobis(3-ethylbenzothiazoline-6-sulfonic acid)) was used as the substrate were as shown in Table 1.

TABLE 1
Enzymological properties of POD

| | |
|---|---|
| Molecular weight (Kd) | 41 (monomer) |
| Optimum pH | 6.0 to 7.0 |
| pH Stability | 7.0 to 11.0 |
| Optimum temperature | 30 to 55° C. |
| Temperature stability | Less than 60° C. |
| Storage stability (liquid) | About 6 months (4° C.) |

4) Labeling (Crosslinking) of Antibody with Maleimidation POD and Purification of Labeled Antibody

The aforementioned maleimidation POD and SH group reduction antibody (solvent is 0.1 M sodium phosphate (pH 6.8), 0.15 M NaCl, 10 mM EDTA) were mixed in such a manner that the enzyme:antibody molar ratio became 10:1. After incubation at 4° C. for 6 hours or more (overnight), the reaction solution after labeling (crosslinking) was subjected to Superdex 200 10/300 (mfd. by GE, 17-1071-01, elution solution: PBS-containing 0.5 M NaCl, column size: about 24 ml, flow rate: 0.5 ml/min) which had been equilibrated with PBS containing 0.5 M NaCl, and the enzyme which was not bounded with the antibody was removed to obtain POD-labeled anti-mouse IgG (POD-labeled antibody of IgG, POD-IgG). Regarding Fab′, POD-labeled anti-mouse Fab′ (POD-labeled antibody of Fab′, POD-Fab′) was also obtained by subjecting to Superdex 200 10/300 GL and removing the enzyme which was not bonded with the antibody.

### Example 2

### Reactivity Comparison Test in the Detection of Mouse IgG (Primary Antibody) by ELISA (Coloring System)

Reactivity comparison in the detection of mouse IgG (primary antibody, prepared for human transferring by a usual method) was carried out using labeled antibodies prepared in accordance with the method of Example 1 or commercially available horseradish (HRP) labeled antibodies.

### 1. Used Antibodies

1) Commercially available horseradish POD-labeled antibody 1 (used in the assay system of Comparative Example 1): Peroxidase-conjugated AffiniPure F(ab′)₂ Fragment Goat Anti-mouse IgG (H+L) (HRP-F(ab′)₂) (mfd. by Jackson ImmunoResearch Labs.)
2) Commercially available horseradish POD-labeled antibody 2 (used in the assay system of Comparative Example 2): Peroxidase-conjugated AffiniPure Goat Anti-mouse IgG (H+L) (HRP-IgG) (mfd. by Jackson ImmunoResearch Labs.)
3) POD-labeled antibody of Fab′ obtained in Example 1 (used in the assay system of Invention 1)
4) POD-labeled antibody of IgG obtained in Example 1 (used in the assay system of Invention 2)

### 2. Detection of Mouse IgG by ELISA (Coloring System)

Using a mouse IgG solution prepared into a concentration of 1, 10 or 100 ng/ml, the mouse IgG (primary antibody: anti-human transferrin monoclonal antibody (mfd. by BIO MATRIX RESEARCH, INC.)) was immobilized on a 96-well microtiter plate in the usual way. The above-mentioned various types of POD-labeled antibodies (secondary antibodies) were respectively added thereto and allowed to undergo the reaction at room temperature for 1 hour. Thereafter, washing was carried out three times with TBS containing 0.05% Tween 20. After the washing, a substrate solution prepared by dissolving ABTS-Tablets (2,2′-azinobis(3-ethylbenzothiazoline-6-sulfonic acid), mfd. by Roche) in ABTS Buffer (mfd. by Roche) was added thereto and allowed to undergo the reaction for a certain period of time, followed by measurement of absorbance at 405.

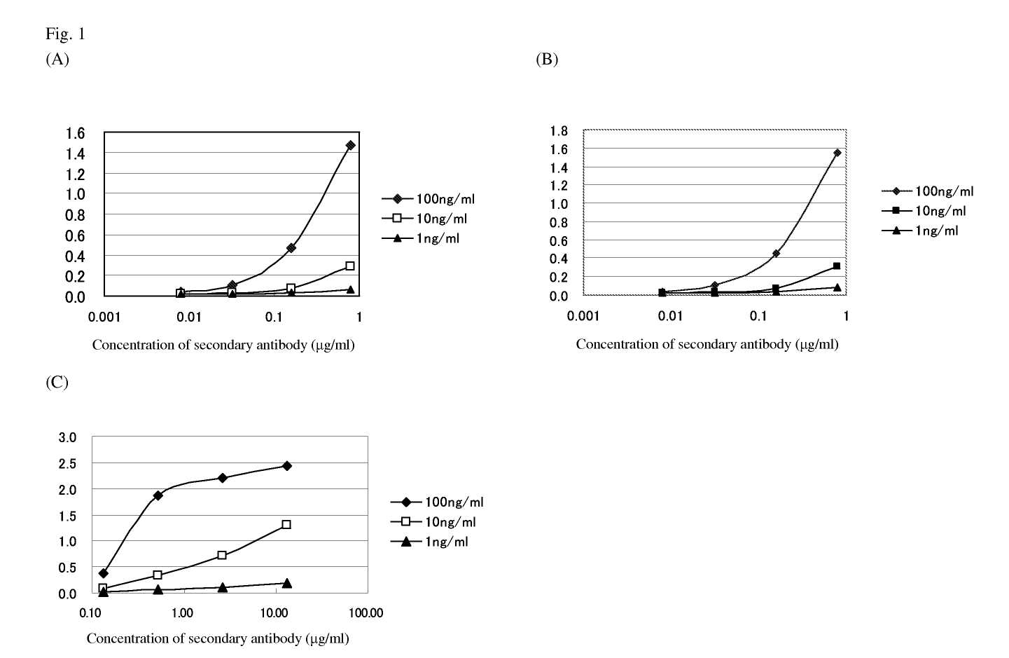

As an example of the measured values, a relationship between the concentration of used secondary antibodies and the coloring level, regarding the POD-labeled antibody of Fab′, is shown in FIG. 1.

As shown in FIG. 1, independent of the concentration of the immobilized mouse IgG, increase of the coloring level was observed in response to the increase of used amount of the POD-labeled antibodies. Additionally, particularly when amount of the measuring control (primary antibody) is relatively large, it is also indicated that even when using amount of the secondary antibody is increased to a certain level or more, there is a limitation to the improving effect of coloring level (it is considered that one of the causes of this is due to limitation of the coloring level measurement).

By the test, it was shown that when similar amount of the mouse IgG is measured using similar amount of the POD-labeled antibody, within a numerical value range which is suitable for the coloring level measurement, the case of using the POD-labeled antibodies of the present invention shows a higher sensitivity than the case of using commercially available horseradish POD-labeled antibodies. For example, in comparison by the absorbance in the case of measuring the immobilized mouse IgG using a solution prepared by using about 0.8 μg/ml of secondary antibody and adjusting to a concentration of 10 ng/ml or 100 ng/ml, it was about 0.3 and about 1.4, respectively, in the case of the use of the horseradish POD-labeled antibody (Comparative Example 1 and Comparative Example 2), while it was about 0.4 and about 1.9 in the case of the use of the POD-labeled antibody of the present invention (Invention 1). Thus, about 1.3 to 1.4 times higher value was obtained.

In the same manner, it was found that good measured values can be obtained also when the POD-labeled antibody prepared using IgG was used (Invention 2).

### Example 3

### Comparison of Reactivity in the Detection of Mouse IgG by Human Transferrin Sandwich ELISA (Coloring System)

A system of sandwich ELISA for measuring human transferrin by detecting mouse IgG by a coloring system was constructed using labeled antibodies prepared in accordance with the method of Example 1 or commercially available horseradish POD-labeled antibodies, and their coloring levels were compared.

### 1. Used Antibodies

1) Commercially available horseradish POD-labeled antibody 1 (used in the assay system of Comparative Example 3): Peroxidase-conjugated AffiniPure F(ab′)₂ Fragment Goat Anti-mouse IgG (H+L) (HRP-F(ab′)₂) (mfd. by Jackson ImmunoResearch Labs.)
2) Commercially available horseradish POD-labeled antibody 2 (used in the assay system of Comparative Example 4): Peroxidase-conjugated AffiniPure Goat Anti-mouse IgG (H+L) (HRP-IgG) (mfd. by Jackson ImmunoResearch Labs.)
3) Commercially available horseradish POD-labeled antibody 3 (used in the assay system of Comparative Example 5): Anti-IgG (H+L), Mouse, Goat-Poly (HRP-IgG) (mfd. by Bethyl)
4) POD-labeled antibody of Fab′ obtained in Example 1 (used in the assay system of the present invention 3)
5) POD-labeled antibody of IgG obtained in Example 1 (used in the assay system of the Invention 4)

### 2. Detection of Mouse IgG by Human Transferrin Sandwich ELISA (Coloring System)

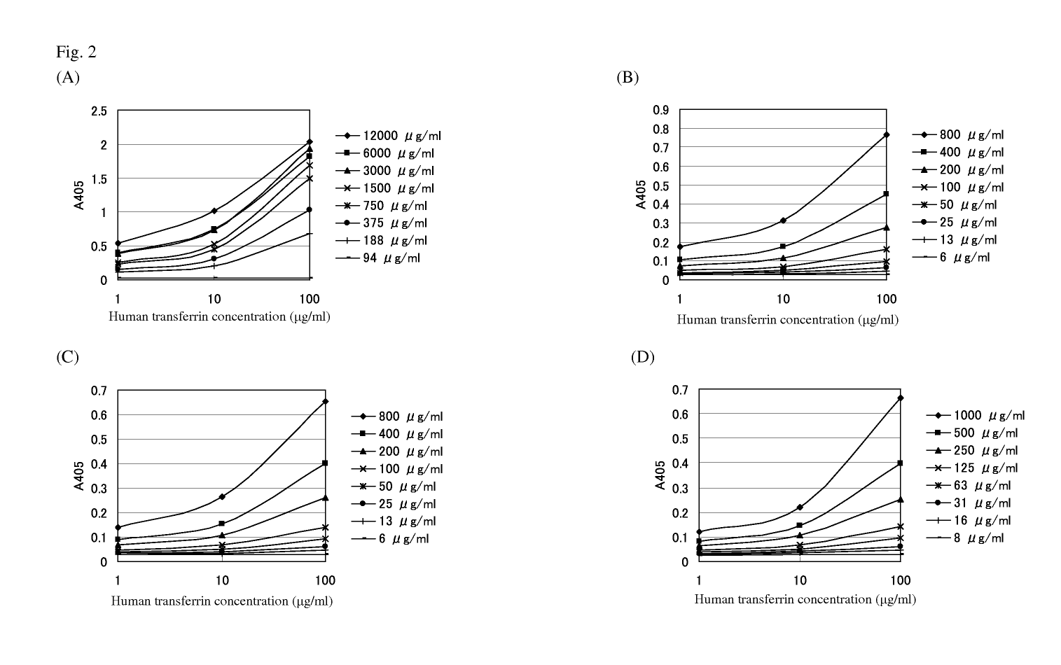

To a 96-well microtiter plate onto which an anti-human transferrin monoclonal antibody (mfd. by BIO MATRIX RESEARCH, INC.) had been immobilized in the usual way, 50 μl portion of a solution containing 1, 10 or 100 ng/ml of transferrin, prepared using PBS was added and allowed to undergo the reaction at room temperature for 1 hour. After the reaction, washing was carried out three times using 350 μl of TBS containing 0.05% Tween 20. After the washing, optionally diluted anti-human transferrin monoclonal antibody for detection was added to each well and allowed to undergo the reaction at room temperature for 1 hour. After the reaction, washing was carried out three times using TBS containing 0.05% Tween 20. After the washing, each of the various POD-labeled antibodies was added thereto and allowed to undergo the reaction at room temperature for 1 hour. After the reaction, washing was carried out three times using TBS containing 0.05% Tween 20. After the washing, a substrate solution prepared by dissolving ABTS-Tablets (2,2′-azinobis(3-ethylbenzothiazoline-6-sulfonic acid), mfd. by Roche) in ABTS Buffer (mfd. by Roche) was added thereto and allowed to undergo the reaction for a certain period of time, and then absorbance at 405 nm (coloring level) was measured. As an example of the measured values, a relationship between the concentration of transferrin and the coloring level, in the coloring system using the HRP-labeled antibody and the POD-labeled antibody obtained in Example 1, is shown in FIG. 2.

As shown in FIG. 2, increase in the coloring level was observed in response to the used amount of each POD-labeled antibody at each concentration of the added human transferrin. Additionally, when the POD-labeled antibodies were used in the same amount, an increasing tendency of the coloring level in response to the increase of human transferrin concentration was confirmed.

In the test, it was shown that when the human transferrin (antigen) is measured using similar amount of the anti-human transferrin monoclonal antibody and using similar amount of the POD-labeled antibodies, within a numerical value range suitable for the coloring level measurement, the case of using the POD-labeled antibodies of the present invention shows a higher sensitivity than the case of using commercially available horseradish POD-labeled antibodies. For example, in comparison by the absorbance in the case of measuring the immobilized mouse IgG using 100 ng/ml of the transferrin solution, it was about 0.45 in the case of the use of the horseradish POD-labeled antibody (400 ng/ml) (Comparative Example 3), while it was about 1.0 in the case of the use of the POD-labeled antibody of the present invention (375 ng/ml) (Invention 3). Thus, about 2.2 times higher value was obtained. Namely, it was found that when labeled antibodies are used in an amount similar to that of the horseradish POD-labeled antibodies, or rather in a smaller amount than that, a higher coloring level is obtained in the case of the use of the POD-labeled antibodies of the present invention.

The above-mentioned tendency was particularly significant regarding the POD-labeled antibody of Fab′, but it was found that a good measured value can be obtained even in the case of the use of the POD-labeled antibody prepared using IgG (Invention 4).

### Example 4

### Reactivity Comparison in the Detection of Mouse IgG by Human Transferrin or α2-Macroglobulin Sandwich ELISA (Coloring System)

A system of sandwich ELISA for measuring human transferrin or α2-macroglobulin by detecting mouse IgG by a coloring system was constructed using labeled antibodies prepared in accordance with the method of Example 1 and commercially available horseradish POD-labeled antibodies, and their coloring levels were compared. Specifically, human transferrin or α2-macroglobulin was measured by a sandwich ELISA and its coloring level was compared in accordance with the method of Example 3, except that, after the addition of the POD-labeled antibodies and subsequent washing, a substrate solution prepared by dissolving luminol (mfd. by Sigma) and hydrogen peroxide in tris (at the time of the use of horseradish POD-labeled antibodies), or in Tricine (at the time of the use of the POD-labeled antibodies of the present invention), was added instead of 2,2′-azinobis(3-ethylbenzothiazoline-6-sulfonic acid) (ABTS) and allowed to undergo the reaction for a certain period of time, followed by measurement of the coloring level. In this connection, although using amounts of the used POD-labeled antibodies were not the same, each case was subjected to a preliminary test and used in an optimized concentration so that proper reaction can be carried out by the assay system.

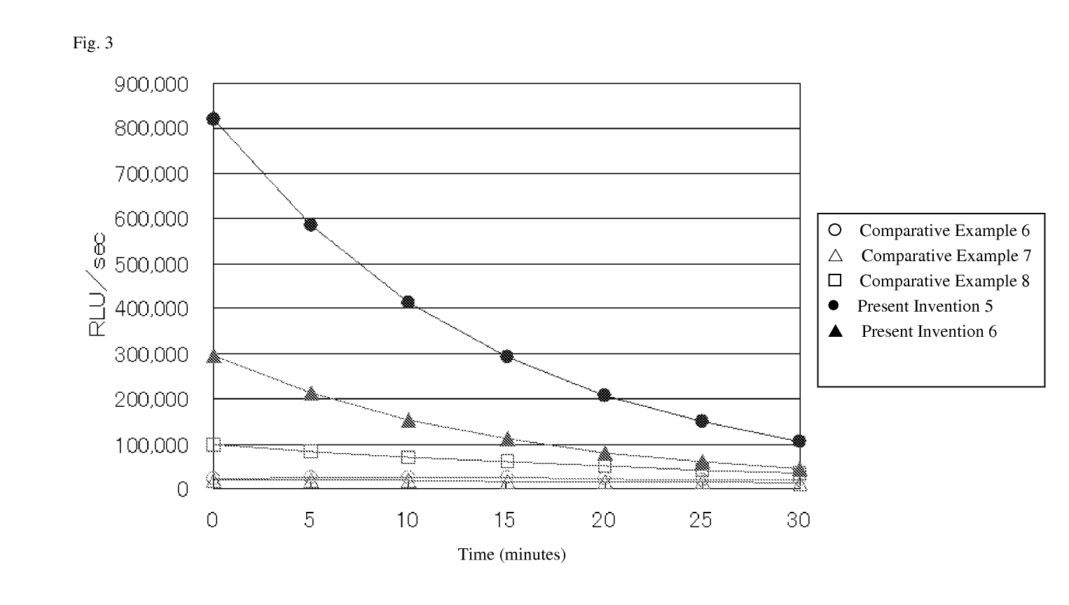

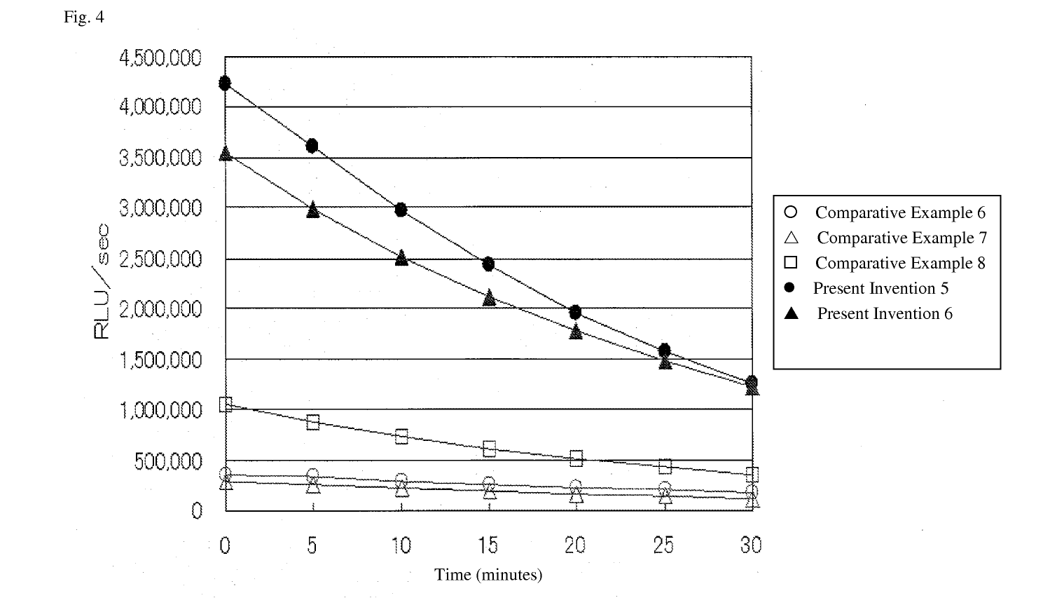

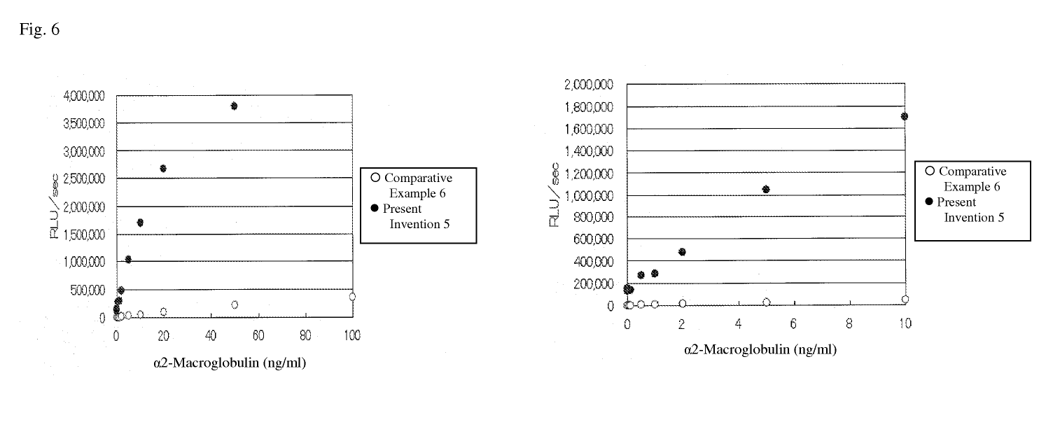

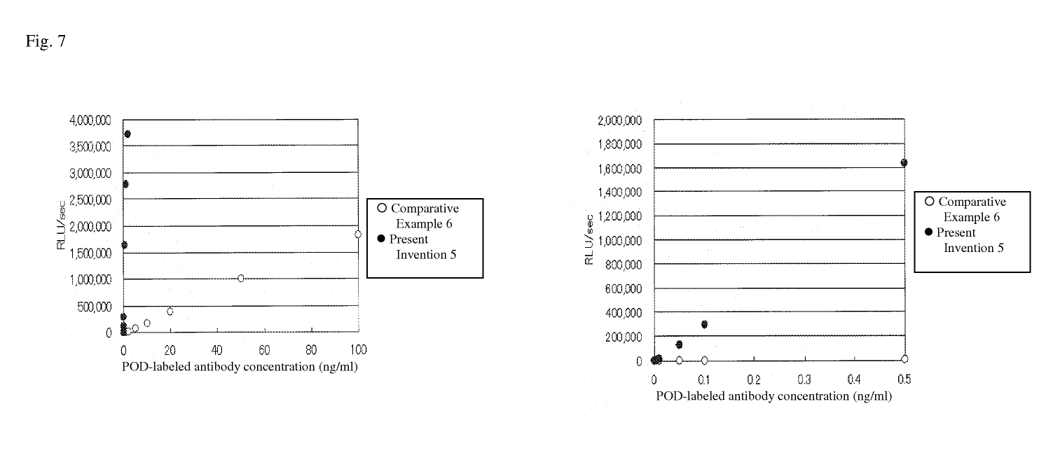

As an example of the measured values, periodical change in the emission intensity when 1 ng/ml of transferrin solution was used is shown in FIG. 3, and periodical change in the emission intensity when 100 ng/ml of transferrin solution was used is shown in FIG. 4, both in the case of using various POD-labeled antibodies. Change in the emission intensity at the time of the addition of reagents when concentration of α2-macroglobulin was varied while fixing the concentration of various POD-labeled antibodies is shown in FIG. 6, and change in the emission intensity at the time of the addition of reagents when the concentration of various POD-labeled antibodies was varied while fixing concentration of α2-macroglobulin is shown in FIG. 7, respectively.

Different from the case of the coloring system in which the absorbance periodically increases during a certain period of time as the coloring reaction advances, in the emission system, the light at the initial stage (just after the emission reaction) is most strong and is periodically reduced thereafter. In the case of the actual measurement of emission level, integrated value of the emission levels during a certain period of time is used in many cases, but the integrated value depends on the initial stage emission level and the degree of its attenuation, particularly depends on the initial stage emission level.

In all of the cases of measuring human transferrin (antigen) at any concentration, it was shown that those which used the POD-labeled antibodies (Inventions 5 and 6) have markedly higher initial stage emission values and also integrated emission values until 30 minutes after the measurement than those which used the commercially available horseradish POD-labeled antibodies (Comparative Examples 6 to 8).

For example, in the case of the initial stage emission value when 1 ng/ml of the transferrin solution was used as shown in FIG. 3, when three kinds of horseradish POD-labeled antibodies were used (Comparative Examples 6 to 8), the initial stage emission values were markedly low. Even the highest value is 100,000 RLU (Comparative Example 7), while it was about 800,000 RLU in the case of the use of the POD-labeled antibody of the present invention (Invention 5), showing 8 times higher value. In the case of the use of the POD-labeled antibodies of the present invention in which IgG was labeled with the POD (Invention 6), although the emission values were relatively low among the two kinds of POD-labeled antibodies of the present invention, the initial stage emission values were still about 300,000 RLU so that 3 times or more higher values than the case of the use of the horseradish POD-labeled antibodies were obtained.

This tendency was the same also in the case of the use of 100 ng/ml of the transferrin solution as shown in FIG. 4. When horseradish POD-labeled antibodies were used (Comparative Examples 6 to 8), the initial stage emission values were about 250,000 RLU and 1,000,000 RLU, while it was about 4,200,000 RLU in the case of the use of the POD-labeled antibody of the present invention (Invention 5), showing 4.5 times and 18 times or more higher value. Even in the case of the use of the POD-labeled antibodies of the present invention labeled on IgG (Invention 6), it became about 3,500,000 RLU so that 3.5 times and 14 times or more higher values than the case of the use of the horseradish POD-labeled antibodies were obtained.

As described in the above, although the high sensitivity of the POD-labeled antibodies of the present invention in the emission system was particularly significant regarding the POD-labeled antibodies of Fab′, it was also excellent in the case of the use of the POD-labeled antibodies prepared using IgG, so that it was found that these are markedly excellent in comparison with the case of the use of horseradish POD-labeled antibodies.

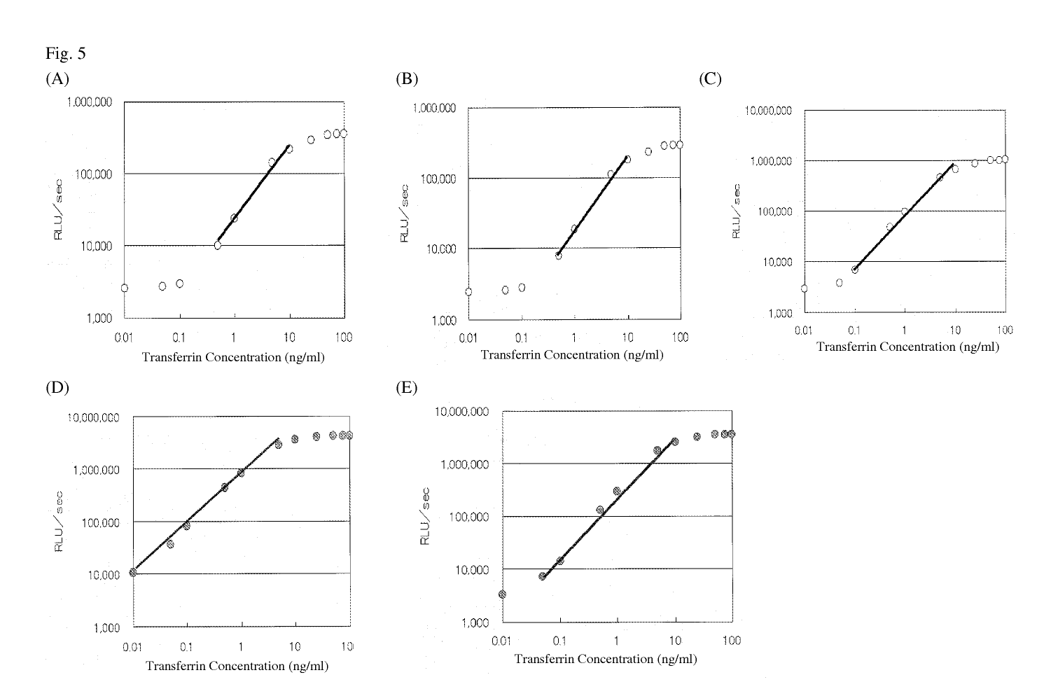

A relationship between the concentration of transferrin and the initial stage emission level, calculated from the data of this measurement, is shown in FIG. 5. The straight line part in each graph of this drawing shows concentration range of transferrin measurable with high accuracy, based on an assumption that concentration of transferrin is calculated using the test system of the measurement. Table 2 shows a result of calculating, from the plotting of respective data, concentration range (measurable concentration range) of transferrin from which a linearity of R²=0.99 or more can be obtained.

TABLE 2

| | Origin of | Kind of | Measurable concen- |
|---|---|---|---|
| | POD used | antibody used | tration range of |
| Assay system | in labeling | in labeling | transferrin (ng/ml) |
| Comparative | Horseradish | F(ab′)₂ | 0.5 to 10 |
| Example 6 | | | |
| Comparative | Horseradish | IgG | 0.1 to 10 |
| Example 7 | | | |
| Comparative | Horseradish | IgG | 0.5 to 10 |
| Example 8 | | | |
| Present | Genus Coprinus | Fab′ | 0.01 to 10 |
| Invention 5 | | | |
| Present | Genus Coprinus | IgG | 0.05 to 10 |
| Invention 6 | | | |

As shown in Table 2, it was shown that a transferrin concentration of lower than 0.5 ng/ml, 0.1 ng/ml in the best case, cannot be measured accurately by the measuring methods of Comparative Examples (horseradish POD-labeled antibodies were used). Contrary to this, it was shown that when the methods of the present invention (a POD derived from the genus Coprinus was used) are used, transferrin can be measured suitably at a low concentration 0.01 ng/ml, 0.05 ng/ml in the worst case.

Additionally, as shown in FIG. 6, when concentration of various POD-labeled antibodies was fixed to a certain appropriate concentration and concentration of α2-macroglobulin (antigen) was varied, significantly high measured values were obtained by those in which the POD-labeled antibody of the present invention was used (Invention 5) than by those in which the commercially available horseradish POD-labeled antibody was used (Comparative Example 6), in the case of measuring every concentration of α2-macroglobulin. It was confirmed that the POD-labeled antibody of the present invention has higher sensitivity than this measured value (shown by an integrated value during 1 second just after the addition of the reagent). In order to explain a difference between the present invention and comparative examples more plainly, concentration of α2-macroglobulin was shown by two drawings of a range of from 0 to 100 ng/ml and a low concentration range of from 0 to 10 ng/ml. Depending on the concentration of the antigen, the POD-labeled antibody of the present invention showed 30 times or more higher sensitivity. By carrying out the same examination on the system of human transferrin, it was confirmed that it also has high sensitivity.

As shown in FIG. 7, when concentration of α2-macroglobulin (antigen) was fixed to a certain appropriate concentration and concentration of various POD-labeled antibodies was varied, significantly high measured values were obtained by those in which the POD-labeled antibody of the present invention was used (Invention 5) than by those in which the commercially available horseradish POD-labeled antibody was used (Comparative Example 6), in the case of every concentration of POD-labeled antibodies. In this connection, other measuring conditions in that case were identical to the case of fixing concentration of POD-labeled antibody and varying concentration of antigen. In order to explain a difference between the present invention and comparative examples more plainly, concentration of α2-macroglobulin was shown by two drawings of a range of from 0 to 100 ng/ml and a low concentration range of from 0 to 0.5 ng/ml. Depending on the concentration of labeled antibodies, the POD-labeled antibody of the present invention showed 200 times or more higher sensitivity. By carrying out the same examination on the system of human transferrin, it was confirmed that it also has high sensitivity.

Based on the above, it was found that it becomes possible to measure a substance of interest using a further smaller number of labeled antibodies and, what is more, in a more smaller amount, by the use of the method of the present invention. Additionally, such a significant improving tendency of reactivity and sensitivity when a POD derived from Basidiomycetes is used, in the case of carrying out an enzyme immunoassay such as ELISA or western blotting using an emission system, was hardly predictable from the reactivity which can be hardly said high when a test is carried out by a system in which the POD derived from Basidiomycetes is not immobilized.

### Example 5

### Preparation of Composition for Enzyme Immunoassay

A composition for enzyme immunoassay was obtained by preparing the POD-labeled reagent, mouse IgG, buffer for dilution, buffer for washing, standard antigen and reagent solution for emission shown in Table 3.

TABLE 3

| Reagent name | Composition |
|---|---|
| 1) POD-labeled | POD-labeled reagent of Example 1: |
| reagent | POD-labeled reagent of Fab′, 156 ng/ml |
| 2) Mouse IgG | Anti-human transferrin monoclonal antibody |
| | (mfd. by BIO MATRIX RESEARCH, INC.), 5000 |
| | times dilution |
| 3) buffer for | PBS (140 mM NaCl, 8.1 mM Na₂HPO₄•12 H₂O, 3 |
| dilution | mM KCl, 1.47 mM KH₂PO₄, pH 7.4) |
| 4) buffer for | 0.05% Tween 20-containing TBS (20 mM Tris-HCl, pH |
| washing | 7.5, 0.15 M NaCl) |
| 5) standard | Human transferrin standard article (mfd. by BIO |
| antigen | MATRIX RESEARCH, INC.) 10 ng/ml |
| 6) reagent | 1.5 mM Luminol (mfd. by Sigma), 0.75 mM hydrogen |
| solution for | peroxide (mfd. by Wako Pure Chemical Industries)- |
| emission | containing 100 mM Tricine, pH 8.8 |

### Example 6

### Preparation of Kit for Enzyme Immunoassay

The 6 types of reagents prepared in accordance with Example 5 were put into respective subdivision containers, and respective reagents and a 96-well microtiter plate were assorted in one set and used as a kit for enzyme immunoassay.

When human transferrin was measured using the kit prepared in the above-mentioned manner, it was able to carry out the measuring operation properly and efficiently, and the results similar to those shown in the Invention 8 of Example 4 were obtained.

### Example 7

### Detection of Human Transferrin Using Western Blotting

To SDS-PAGE, a 100 ng portion of human-derived plasma and 10 ng of human transferrin were subjected. After the completion thereof, the gel was transferred onto a PVDF film by western blotting. The transferred film was washed in the usual way and subjected to blocking, and an anti-human transferrin monoclonal antibody was added thereto, followed by the reaction at room temperature for 1 hour. After the reaction, washing is carried out for three times with TBS containing 0.05% Tween-20, and two kinds of labeled antibodies (POD-IgG and POD-Fab′) prepared in accordance with the method of Example 1 were optionally diluted and added thereto, followed by the reaction at room temperature for 1 hour. After the reaction, washing was carried out for three times with TBS containing 0.05% Tween 20. Subsequently, an emission reagent containing luminol (ECL Detection Reagent (mfd. by GE)) was added thereto and the emission was detected.

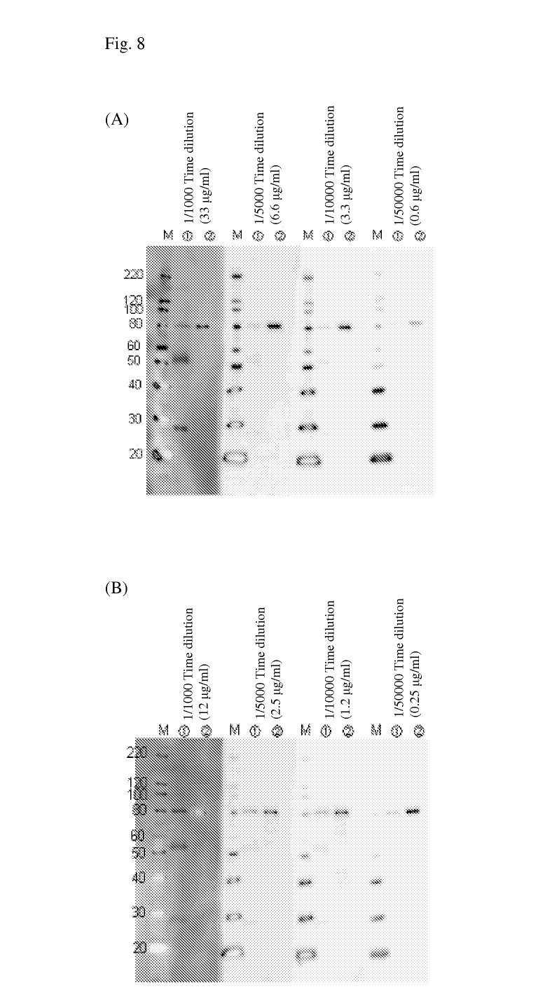

The results are shown in FIG. 8. In the drawing, the case shown as “1” (enclosed letter of a circle) is a result of 100 ng of plasma, and the case shown as “2” (enclosed letter of a circle) is a result of 10 ng of human transferrin. M is a molecular weight marker. In either case of the use of the labeled antibodies, both were able to be detected as respective bands properly with no problems so that it was shown that the labeled antibodies of the present invention can be suitably used in the western blotting.

### Example 8

### Immunostaining of HeLa Cell by Anti-Actin Antibody

Using the above-mentioned three types of POD-labeled antibodies (IgG, Fab′) to be used in the present invention, immunostaining of HeLa cell by an anti-actin antibody was carried out. The immunostaining was carried out in accordance with the general method described in, for example, an antibody experimentation manual published by Yohdo-sha, and the like. As the primary antibody, an anti-actin monoclonal antibody was used. As the second antibody, the POD-labeled antibodies prepared by the method of Example 1 were respectively added in appropriate amounts.

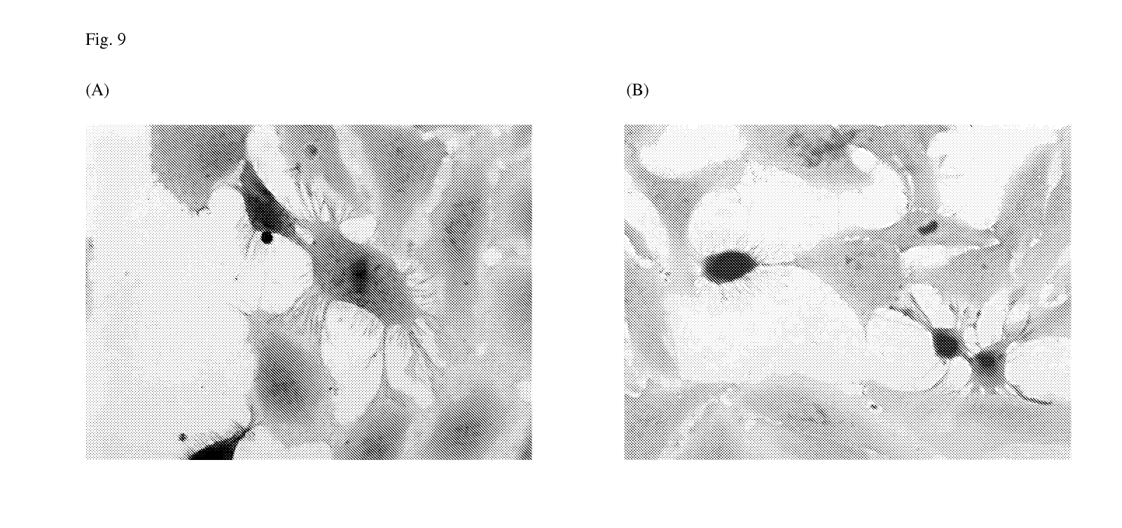

An example of the results is shown in FIG. 9. Regarding either system, a region which corresponds to the existing region of actin in the cell was stained, thus showing that the POD-labeled antibodies to be used in the present invention can be used suitably.

### Example 9

### Immunostaining of Rat Pancreas by Anti-Insulin Antibody

Using the above-mentioned two types of POD-labeled antibodies (IgG, Fab′), immunostaining of rat pancreas by an anti-insulin antibody was carried out. The immunostaining was carried out in accordance with the general method described in, for example, an antibody experimentation manual published by Yohdo-sha, and the like. As the primary antibody, an anti-insulin monoclonal antibody was used. As the second antibody, the labeled antibodies prepared by the method of Example 1 were diluted to 1/1000 and then added.

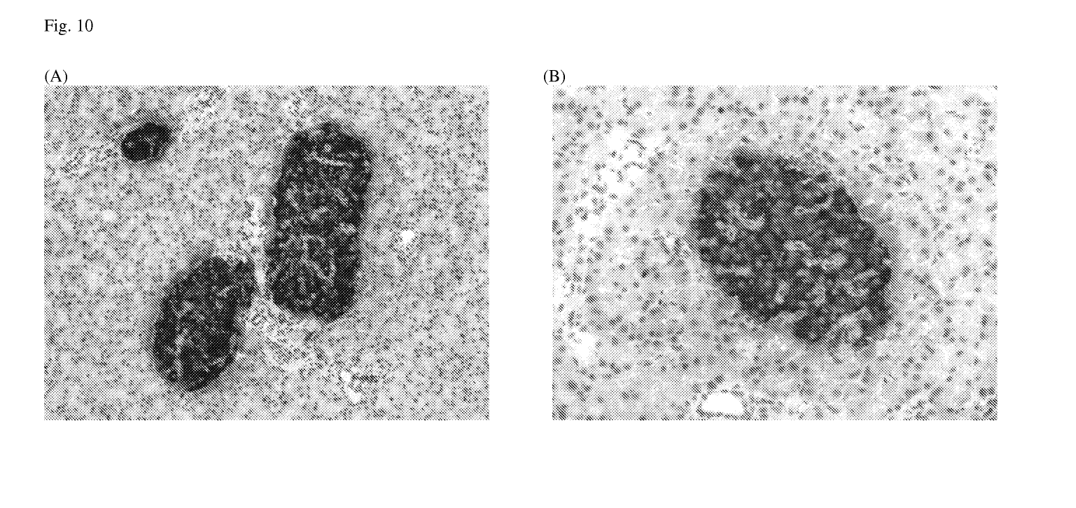

An example of the results is shown in FIG. 10. Regarding either system, a region which corresponds to the existing region of insulin in the cell was stained, thus showing that the POD-labeled antibodies to be used in the present invention can be used suitably.

### Example 10

### Confirmation of Influence of the Coexistence of Sodium Azide Upon POD Activity Value

A labeled antibody prepared in accordance with the method of Example 1 or a commercially available horseradish POD-labeled antibody was allowed to coexist with from 0 to 1% of sodium azide (mfd. by Wako Pure Chemical Industries) for a certain period of time (3 hours), and then comparison of reactivity in the detection of mouse IgG (primary antibody, prepared by a general method for human transferrin) was carried out. The measuring method is as in Example 2.

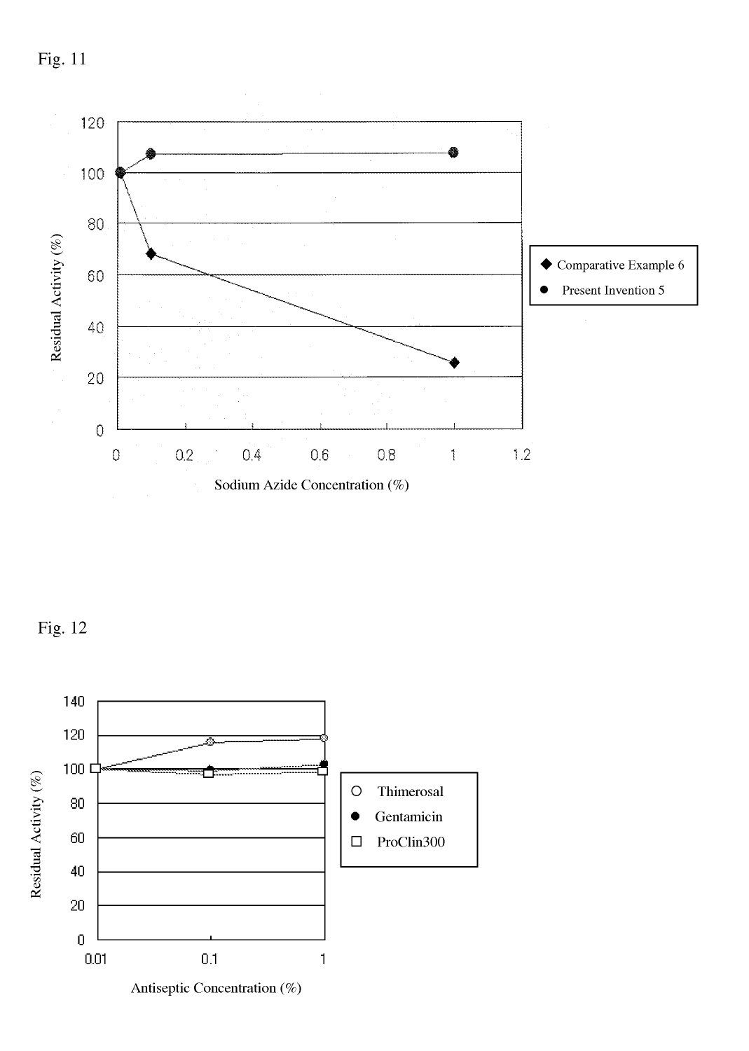

An example of the results is shown in FIG. 11. Although when the horseradish POD-labeled antibody (Comparative Example 6) was used, the POD activity was sharply inhibited depending on the concentration of coexisting sodium azide, when the POD-labeled antibody of the invention (Invention 5) was used, inhibition of the POD activity was not observed even under the coexistence of sodium azide so that its excellent antiseptic resistance was confirmed. Based on this, it was shown that the composition for immunoassay of the present invention can suitably preserve and use the POD-labeled antibodies without particularly requiring an additive even under the coexistence of sodium azide.

### Example 11

### Confirmation of Influence of the Coexistence of Various Antiseptics Upon POD Activity Value

Using a member of the universally used proclin, Proclin 300 (mfd. by Sigma), thimerosal (mfd. by Sigma) and gentamicin (mfd. by Wako Pure Chemical Industries) as additional antiseptics other than Example 10, antiseptic resistance was tested by carrying out the same operation of Example 10.

An example of the results is shown in FIG. 12. In all of the antiseptics, inhibition of the POD activity was not found by the measurement using the composition for immunoassay of the present invention. Namely, it was shown that the composition for immunoassay of the present invention can be suitably preserved and used without spoiling the POD activity in carrying out the measurement even when various antiseptics are used.

According to the present invention, it is able to provide a method by which an enzyme immunoassay can be carried out with high reactivity and sensitivity similar or superior to the case of using horseradish POD-derived POD, even when the horseradish POD-derived POD is not used. Additionally, even when antiseptics including sodium azide are added, the POD activity is hardly inhibited so that an enzyme immunoassay such as ELISA can be carried out with good reactivity and sensitivity.

While the invention has been described in detail and with reference to specific embodiments thereof, it will be apparent to one skilled in the art that various changes and modifications can be made therein without departing from the spirit and scope of the invention.

This application is based on a Japanese patent application filed on Jul. 15, 2008 (Japanese Patent Application No. 2008-183414) and a Japanese patent application filed on Jul. 13, 2009 (Japanese Patent Application No. 2009-164343), the entire contents thereof being thereby incorporated by reference.

What is claimed is:

1. An enzyme immunoassay, which comprises measuring a coloring level or emission level derived from a peroxidase reaction by an antibody or fragmentation antibody which is labeled with a peroxidase derived from Basidiomycetes, wherein said coloring level or emission level is increased in comparison with the case of using an antibody or fragmentation antibody which is labeled with a peroxidase derived from horseradish.

2. The enzyme immunoassay according to claim 1, wherein the antibody or fragmentation antibody is immobilized.

3. The enzyme immunoassay according to claim 1, wherein the Basidiomycetes is a microorganism belonging to the genus Coprinus.

4. The enzyme immunoassay according to claim 1, wherein the peroxidase derived from Basidiomycetes is a polypeptide having an amino acid sequence wherein at least one of deletion, addition, insertion and substitution is made on one or two or more amino acid residues of the amino acid sequence described in SEQ ID NO:2.

5. The enzyme immunoassay according to claim 1, wherein the peroxidase derived from Basidiomycetes is a polypeptide having an amino acid sequence described in SEQ ID NO:2.

6. The enzyme immunoassay according to claim 1, wherein the peroxidase derived from Basidiomycetes or an antibody or fragmentation antibody labeled with the peroxidase derived from Basidiomycetes has an antiseptic resistance.

7. The enzyme immunoassay according to claim 6, wherein the antiseptic is proclin, thimerosal, sodium azide, gentamicin or a derivative thereof.

8. A composition for enzyme immunoassay, which comprises an antibody labeled with a peroxidase derived from genus Coprinus, wherein the aforementioned labeled antibody carries out a coloring reaction or emission reaction under an immobilized state.

9. The composition for enzyme immunoassay according to claim 8, wherein the peroxidase derived from genus Coprinus or an antibody or fragmentation antibody labeled with the peroxidase derived from genus Coprinus has an antiseptic resistance.

10. The composition for enzyme immunoassay according to claim 9, wherein the antiseptic is proclin, thimerosal, sodium azide, gentamicin or a derivative thereof.

11. A kit for enzyme immunoassay, which comprises the composition for enzyme immunoassay described in claim 8.

12. An antibody or fragmentation antibody labeled with a peroxidase derived from a Basidiomycete belonging to the genus Coprinus.

13. The antibody or fragmentation antibody according to claim 12, which is a polypeptide having the amino acid sequence described in SEQ ID NO:2.


SEQUENCE LISTING (2 sequences)

SEQ ID NO: 1 - DNA, 1092 bp, Coprinus cinereus
atgaagctctcgcttttatccaccttcgctgctgtcatcatcggtgccctcgctctcccccagggtcctggaggaggcgggtcagtcacttgccccggtggacagtccacttcgaacagccagtgctgcgtctggttcgacgttctagacgatcttcagaccaacttctaccaagggtccaagtgtgaaagccctgttcgcaagattcttcgaattgttttccatgacgcgatcggattttcgccggcgttgactgctgctggtcaattcggtggtggaggagctgatggctccatcattgcgcattcgaacatcgaattggccttcccggctaatggcggcctcaccgacaccatcgaagccctccgcgcggtcggtatcaaccacggtgtctctttcggcgatctcatccaattcgccactgccgtcggcatgtccaactgccctggctctccccgacttgagttcttgacgggcaggagcaacagttcccaaccctcccctccttcgttgatccccggtcccggaaacactgtcactgccatcttggatcgtatgggcgatgcaggcttcagccctgatgaagtcgttgacttgcttgctgcgcatagtttggcttctcaggagggtttgaactcggctatcttcaggtctcctttggactcgacccctcaagttttcgatacccagttctacattgagaccttgctcaagggtaccactcagcctggcccttctctcggctttgcagaggagctctcccccttccctggcgaattccgcatgaggtccgatgctctattggctcgcgactcccgaaccgcctgccgatggcaatccatgaccagtagcaatgaagttatgggccagcgataccgcgccgccatggccaagatgtctgttctcggcttcgacaggaacgccctcaccgattgctctgacgttattccttctgctgtgtccaacaacgctgctcctgttatccctggtggccttactgtcgatgatattgaggtttcgtgcccgagcgagcctttccctgaaattgctaccgcctcaggccctctcccctccctcgctcctgctccttga

SEQ ID NO: 2 - protein, 363 aa, Coprinus cinereus
MKLSLLSTFAAVIIGALALPQGPGGGGSVTCPGGQSTSNSQCCVWFDVLDDLQTNFYQGSKCESPVRKILRIVFHDAIGFSPALTAAGQFGGGGADGSIIAHSNIELAFPANGGLTDTIEALRAVGINHGVSFGDLIQFATAVGMSNCPGSPRLEFLTGRSNSSQPSPPSLIPGPGNTVTAILDRMGDAGFSPDEVVDLLAAHSLASQEGLNSAIFRSPLDSTPQVFDTQFYIETLLKGTTQPGPSLGFAEELSPFPGEFRMRSDALLARDSRTACRWQSMTSSNEVMGQRYRAAMAKMSVLGFDRNALTDCSDVIPSAVSNNAAPVIPGGLTVDDIEVSCPSEPFPEIATASGPLPSLAPAP